(19) United States
(12) Patent Application Publication
(10) Pub. No.: US 2023/0414665 A1
(43) Pub. Date: Dec. 28, 2023

(54) METHOD FOR PRODUCING MODIFIED MESENCHYMAL STROMAL STEM CELLS WITH IMPROVED PROPERTIES, MODIFIED CELLS OBTAINED BY THIS METHOD, COMPOSITION INCLUDING SUCH CELLS

(71) Applicant: Centro Di Riferimento Oncologico, Aviano (IT)

(72) Inventors: Mario Mazzucato, Concordia Sagittaria (IT); Francesco Agostini, Budoia (IT); Cristina Durante, Oderzo (IT); Carla Vicinanza, Sellia Marina (IT)

(21) Appl. No.: 18/038,315

(22) PCT Filed: Dec. 13, 2021
(86) PCT No.: PCT/IB2021/061620

§ 371 Date: May 23, 2023

(30) Foreign Application Priority Data
Dec. 14, 2020 (IT) 102020000030692

(51) Int. Cl. A61K 35/28; C12N 5/0775; C12N 13/00

(52) U.S. Cl. CPC A61K 35/28; C12N 5/0667; C12N 13/00; C12N 2501/135; C12N 2509/00; C12N 2533/30

(57) ABSTRACT

The present invention relates to a non-viral method for the production of modified mesenchymal stem/stromal cells comprising the use of an additive rich in human-derived platelet growth factors, derived from platelet concentrates, aimed to promote the expansion of said cells. The method further comprises the phase of submitting the cells to electroporation and suspending said mixture in the growth-medium enriched in human-derived platelet growth factors and keeping in incubation. In this way, it is possible to achieve a chemical-physical, non-viral method in compliance with the Good Manufacturing Practice Guidelines (GMP) for advanced cell therapy products, allowing transiently or stably transfect/deliver gene constructs based on electric pulses administration to cells. Said production method is particularly simple and advantageous in terms of productivity and cost-effectiveness, when compared to methods involving viral vectors.

### INVENTION FIELD

This invention relates to a method for the production of modified mesenchymal stem/stromal cells, to obtain ameliorated properties; the invention is also referred to cells obtained by this method, the composition containing such cells and their use. In particular, the present invention relates to a method to obtain mesenchymal stem/stromal cells genetically modified by a non-viral approach, facilitating the transition of the production pipeline to clinical applications.

### STATE OF ART DESCRIPTION

In the state of the art is known that adipose tissue mesenchymal stem/stromal cells (ASC) can be purified from the stromal vascular fraction (SVF) of the adipose tissue by in vitro cell expansion. Said cells can grow as fibroblasts and they are characterized by capabilities of self-renewal, high proliferation and differentiation toward selected cell types as, but not limited to, adipocytes, chondrocytes, osteocytes as well as cells of neural epithelial and muscle lineage. Said cells can be identified analyzing selected cell surface markers. In particular, ASC are required to highly express CD73, CD90 and CD105 (mesenchymal markers), with concomitant lack of expression of CD11b, CD19, CD45 and HLA-DR.

It was previously characterized, by the state of art in Agostini et al., Stem Cell Research and Therapy 9, 130 (2018), a procedure for the ASC expansion taking advantage of a supernatant rich in growth factors (SRGF) as medium additive derived from human platelet concentrates collected by plasma-platelet apheresis. SRGF additive was previously demonstrated to be an optimal substitute for the fetal bovine serum (FBS) and it was shown to increase 2-3 fold ASC growth rate without affecting differentiation capacity, identity and karyotype stability. In the state of art, mesenchymal stem/stromal cells are known to be characterized by the naive capacity to reach the cancer target after intravenous/systemic administration (homing). In virtue of such feature, mesenchymal stem/stromal cells can be used as targeted delivery vehicles carrying cytotoxic compounds (drug delivery). Nevertheless, only a small fraction of cells can actually reach the neoplastic target. Genetic modification can be considered as a reliable approach to modulate biological properties of said cells, ameliorating on-target homing and cytotoxic properties. Viral transduction approaches to genetically engineer such cells were demonstrated to be highly efficient, but transduced cell products cannot be easily translated to the clinical practice due to insertion mutagenesis risk, as well as, to possible undesired patient (recipient) immune response. In addition, viral cell transduction implies extremely high production costs, determined by viral vector development and by demanding environmental requirements of the cell manipulation facility in compliance with Good Manufacturing Practice (GMP) for advanced cell therapy production. Thus, the Applicant found the need to develop a non-viral method to modify mesenchymal stem/stromal cells to play an efficient cytotoxic effect against targeted cancer cells.

Thus, the principal aim of this invention is to develop a method to obtain ASC characterized by such ameliorated properties.

### INVENTION SUMMARY

A first aspect of the present invention relates to a method, as indicated in claim number 1.

The Applicant of the present patent application, in fact, has surprisingly found that the above-mentioned technical issue can be effectively and affordably solved adopting a mesenchymal stem/stromal cell modification method that involves the following steps:
a) seeding said cells on a plastic support in presence of an appropriate growth-medium promoting their expansion;
b) adding at least one medium additive rich in human-derived platelet growth factors, aimed to promote the expansion of said cells;
c) detaching said expanded cells from the plastic surface;
d) adding to said cells a gene construct, in order to obtain a mixture. Moreover, the method of the present invention also comprises the following phases:
e) submitting said mixture to one or more electrical pulses (electroporation);
f) suspending said electroporated mixture in said growth-medium enriched in human-derived platelet growth factors, and transferring the resulting mixture into a suitable container for incubation;
g) resuspending said electroporated cell mixture in an appropriate container adding an amount of growth-medium containing a percentage of human-derived platelet growth factor additive which is higher than the same growth-medium used in the previous step (b)
h) transferring said electroporated cell mixture to a tissue culture plastic support and incubate in appropriate conditions;
i) at the end of the incubation adding to said container containing genetically modified electroporated cell mixture a volume of growth-medium devoid of said additive rich of human-derived platelet growth factors to restore the equivalent additive concentration applied in the growth-medium used in said phase (b) for cell expansion.

In this way, it is possible to achieve a chemical-physical, non-viral method to transiently or stably transfect/deliver gene constructs: said method is based on electric pulses administration to expanded cells and it is compliant with Good Manufacturing Practice Guidelines (GMP). Said production method is particularly simple and advantageous in terms of productivity and cost-effectiveness, when compared to methods involving viral vectors.

In fact, electroporated cell incubation in a growth-medium particularly rich in human derived platelet growth factors can ameliorate cell viability. Thus, said cells can be used as targeted delivery vehicles carrying cytotoxic compounds against cancer cells. Modification of said mesenchymal stem/stromal cells is required in order for them to exert a cytotoxic effect against target cancer cells.

It is, therefore, possible to induce the expression of factors with potential cytotoxic activity against primary/metastatic cancer cells/masses after autonomous mesenchymal stem/stromal cell homing upon systemic or localized administration.

It is also possible to induce the contemporary expression of one or more protein factors increasing the migration and homing capacity of said cells, in order to improve effectiveness of such targeted delivery vehicles carrying cytotoxic factors against cancer cells.

By a preferred embodiment, said cells were seeded in culture in a growth-medium on a plastic support at appropriate cell density (e.g., around 1.000 cells/cm²) to allow spontaneous cell growth for at least 3 days, avoiding confluence between cells before collection.

By a preferred embodiment, the growth medium is “Minimum Essential Medium Eagle-Alpha Modification (α-MEM)”, even though also other formulations, specifically tailored for mesenchymal stem/stromal cells expansion or adhering fibroblastoid cells, can be adopted.

By a preferred embodiment, said mesenchymal stem/stromal cells are isolated from adipose tissue or other sources e.g., adult tissues and fetal annexes selected from the group consisting of bone marrow, dental pulp, umbilical cord blood, Wharton jelly, amniotic fluid, et similia.

By a preferred embodiment, said medium additive enriched in human-derived platelet growth factors, is herein defined as SRGF and it was previously deeply characterized in Agostini et al., Stem Cell Research and Therapy 9, 130 (2018).

By a preferred embodiment, the medium additive rich of human-derived platelet growth factors is obtained from whole blood or by plasma-platelet apheresis procedures.

By a preferred embodiment, growth factor enrichment within said additive rich in human-derived platelet growth factors is obtained by a physical approach e.g., thermal stress, freeze and thaw, sonication, or by coagulation triggering following the addition of CaCl₂ and/or thrombin.

By a preferred embodiment, said human-derived additive rich of platelet growth factors used in phases b) is added in percentages comprised within a range of 1% to 20% vol/vol, preferably 1.25% to 15% vol/vol, even more preferably 2.5% to 12% vol/vol, most preferably around 5% vol/vol.

By a preferred embodiment, in said step c), said expanded cells are detached from said plastic growth support, following standard cell culture procedures, for example as detailed in the publication of Agostini et al., Stem Cell Research and Therapy 2018.

By a preferred embodiment, after said expanded cells were detached from said plastic support, an appropriate amount of cells, e.g. 500×10³ cells for each electroporation-nucleoporation test, is resuspended in a commercially available buffer.

By a preferred embodiment, an appropriate amount (e.g. 2 μg) of a selected gene construct is added to said resuspension obtaining a mixture of cells and gene construct.

By a preferred embodiment, said gene construct to be delivered into the cell is a eukaryotic expression vector for mammalian cells encoding for green fluorescent protein—GFP, or a eukaryotic expression vector encoding for a protein of interest, in fusion with green fluorescent protein—(GFP), and consists of DNA.

By a preferred embodiment, said mammalian expression vector codes for the cytosolic protein named perforin

By a preferred embodiment, gene construct permeating mesenchymal stem/stromal cells can be the “transposone—sleeping beauty” system, aimed to the stable expression of the gene of interest, with limited risk of insertional oncogenesis.

By a preferred embodiment, said gene construct consists of small and/or micro RNA or it is a vector encoding for said small and/or micro RNA, not directly coding for proteins but interfering and/or regulating gene expression with a direct/indirect activity on the cancer target.

By a preferred embodiment, said cell and gene construct mixture is transferred to appropriate containers, also defined as “electroporation cuvettes” to undergo the administration of electric pulses (electroporation phase).

By a preferred embodiment, said electric pulses are of different intensity and wave shape, aimed to induce multiple transient pore formation in cell membranes through which said gene construct can permeate inside the cell, with reduced impact on cell viability.

By a preferred embodiment, after electroporation, in said step f) electroporated cell mixture is incubated at about 37° C. for a period of less than 60 minutes, for instance about 40 minutes, or preferably for about 20 minutes.

By a preferred embodiment, in said step f) said electroporated cell mixture is incubated in a centrifuge tube not-allowing inside cell adhesion.

By a preferred embodiment, said medium additive enriched in human-derived platelet growth factors used in the above-mentioned step g) is added in percent concentrations nearly twice as high as concentrations used in step b), preferably around 10% vol/vol if in step b) 5% vol/vol was used.

By a preferred embodiment, in said step h) said cells are at first transferred on a tissue culture plastic surface for cell expansion and thereafter incubated at around 37° C. for 60 minutes or less.

By a preferred embodiment, at the end of said incubation phase, in said step i) an equal volume of growth-medium devoid of human-derived platelet growth factors is added to the cell mixture.

By a preferred embodiment, relatively to the addition of 2 ml of complete growth-medium containing 10% SRGF for cell resuspension, addition of 2 ml of growth factor free growth-medium reconstitutes 5% SRGF concentration in a final volume of approximately 4 ml. In this way, growth factor concentration is reconstituted to the original conditions used for cell expansion.

By a preferred embodiment, at the end of said incubation phase, after 12-48 hours from gene transfection, cells are analysed for gene construct expression and transfection efficiency evaluation.

In a second aspect, the present invention refers to mesenchymal stem/stromal cells expanded taking advantage of a growth-medium containing an additive rich of human-derived platelet growth factors, as detailed in claim 8.

In particular, in this second aspect the present invention refers to mesenchymal stem/stromal cells expanded taking advantage of a growth-medium containing an additive rich of human-derived platelet growth factors, obtained by the above-mentioned method, with reference to the first aspect of the present invention.

The obtained mesenchymal stem/stromal cells are characterized by ameliorated global vitality, by an excellent capacity to reach inflammatory/tumor targets (homing) after systemic infusion in circulating blood and/or after static/local administration, representing technical, regulatory and economical advantages, facilitating production pipeline translation to clinical applications.

In a third aspect, the present invention refers to the use of said mesenchymal stem/stromal cells, as indicated in claim 9.

In particular, in this third aspect, the present invention refers to the use of said mesenchymal stem/stromal cells, expanded by a growth-medium containing an additive rich of human-derived platelet growth factors, with reference to the second aspect of the present invention and genetically modified for cytotoxic effect induction against a tumoral mass.

By a preferred embodiment, the types of cytotoxic factors (secreted or membrane-expressed) that can be ectopically over-expressed are: proinflammatory cytokines, anti-angiogenetic factors, factors promoting cell cycle arrest or cell death for necrosis/apoptosis, and factors inhibiting/limiting metastatic or drug resistance capacities of cancer cells, as well as drug precursor converting enzymes (gene-directed enzyme prodrug therapy). The expression of such factors can be inducible and or transient/stable.

By a preferred embodiment, factors for which ectopic over-expression can be induced to improve the cell's homing capabilities include receptors for endothelial adhesion factors that are typically expressed in the activated/inflamed endothelium, receptors of cytokines/chemokines or antigens specifically expressed by the target cancer cell as well as factors directly involved in the physiologic process of interstitial cell migration.

In a fourth aspect, the present invention refers to cell use as indicted in claim 10.

In particular, in this fourth aspect, the present invention refers to the use of mesenchymal stem/stromal cells expanded by a growth-medium containing an additive rich in human-derived platelet growth factors, with reference to the second aspect of the present invention, to induce the expression of trophic factors, anti-inflammatory and/or antioxidant factors, to promote tissue regeneration.

By a preferred embodiment, the invention refers to the contemporary expression of one or more factors promoting migration and/or specific homing properties of cells to ameliorate reconstructive/regeneration potential of such vehicles/vectors.

It is in fact possible to permeate mesenchymal stem/stromal cells with non-coding/interfering small and/or micro RNA with direct or indirect effect on gene regulation or with direct or indirect effect on the damaged tissue.

In a fifth aspect, the present invention refers to the use of mesenchymal stem/stromal cells as indicated in claim 11.

In particular, in this fifth aspect, the present invention refers to the use of such mesenchymal stem/stromal cells expanded by a growth-medium containing an additive rich in human-derived platelet growth factors, with reference to the second aspect of the present invention, and genetically modified for the treatment of pulmonary fibrosarcoma and glioblastoma cells.

In a sixth aspect, the present invention refers to a composition as indicated in claim 12.

In particular, in this sixth aspect, the present invention refers to a composition comprising mesenchymal stem/stromal cells expanded taking advantage of a growth-medium containing an additive rich of human-derived platelet growth factors, obtained by the above-mentioned method, with reference to the first aspect of the present invention.

By a preferred embodiment, said composition comprises said cells in aqueous solution or as scaffold (typically biocompatible polymeric nanocomposite material).

Further system advantages and features of the present invention will become clearly evident from the following detailed description of a preferred but not exclusive embodiment, illustrated by way of non-limiting examples,

### DETAILED DESCRIPTION OF THE INVENTION

The following detailed description refers to a preferred embodiment of a method of the present invention.

### EXAMPLE 1 OF THE INVENTION

50×10³ mesenchymal stem/stromal cells isolated from adipose tissue (ASC) were seeded in T-75 tissue culture flask (75 cm²) (BD Biosciences; Franklin Lakes, NJ, US) in 15 ml of growth-medium of the type Minimum Essential Medium Eagle-Alpha Modification (α-MEM) (Lonza; Basel, Switzerland).

In other embodiments of the present invention it is possible to use other commercially available formulations specifically dedicated to the expansion of mesenchymal cells or of adhesion growing fibroblastoid cells. The number of T-75 flask or different sizes can be selected, based on the required final number of cells to be transfected.

Cells were added with 5% (vol/vol) additive rich of human-derived platelet growth factors (SRGF) extracted from platelets collected by plasma-platelet apheresis.

The cell expansion procedures involving SRGF addition were performed in aseptic conditions under a Class II laminar flow safety cabinet (sterile cabinet).

Expanded cells were placed in a 5% CO2 incubator at 37° C. for at least 3 days. Cells can be left in the incubator without medium exchange or, if necessary, a half volume medium change can be performed before the end of the incubation time.

Cells were collected before contiguous cell-cell contact (confluence) was achieved, as this could limit transfection efficiency.

ASC were detached from plastic surface, after double wash with phosphate buffered saline (PBS), by adding 1 ml of undiluted trypsin-ethylenediaminetetraacetic acid 10× at 37° C. (TrypLe Select; Life Technologies-Thermo Fisher Scientific, Waltham, MA, USA).

ASC containing flasks were placed back in the incubator at 37° C. for 2 minutes until cells were detached, as assessed by microscope observation.

Trypsin activity was neutralized adding 4 ml of complete growth medium (SRGF-containing) and the whole cell mixture was collected.

Cells were concentrated using a standard bench centrifuge (1200 RPM for 5 minutes). Thereafter, cells were counted by a manual hemacytometer (Burker chamber) or by a validated automated device. For each transfection procedure 500×10³ cells were collected and cells were concentrated by using the standard bench centrifuge (1200 RPM for 5 minutes).

Cells were resuspended in NF buffer (Ingenio® Electroporation Solution, Mims Bio Corporation, Madison; USA) (500×10³ cells in 100 μl buffer).

It was added to the cell's mixture 2 μg of the expression vector encoding for the green fluorescent protein—GFP (even if other vectors and genes can be selected in other embodiments); the concentration of the original expression vector stock solution was within the range of 0.5-1 μg/μL in pure water. The mixture was prepared taking advantage of high quality plasmid kit and purification procedures.

The cell mixture was then transferred into the appropriate electroporation cuvette (Ingenio®) where it was subjected to electric pulse administration by the C17 program, using the Nucleofector® 2b device (Lonza).

Immediately after electroporation phase, 0.5 ml of complete medium were added into the electroporation cuvette. Using the appropriate pipette (Ingenio®), cells were transferred to a clean centrifugation tube where they were incubated at 37° C. for 20 minutes.

Cells were then resuspended in the centrifuge tube by gentle pipetting and divided in two containers (wells) of a 6-well plate (BD) previously filled with 2 ml of warm complete medium (37° C.), with twice the concentration (10% vol/vol) of supernatant containing high amounts of growth factors (SRGF) than the concentration (5% vol/vol) of the same SRGF used to expand the cells.

Cells were then placed in a 5% CO₂ incubator at 37° C. for nearly 1 hour.

At the end of the incubation, an equal volume of growth medium without additive rich of human-derived platelet growth factors (SRGF) was added to equilibrate the final growth factor concentration at the optimal condition for cell expansion (e.g., relatively to the addition of 2 ml of complete medium containing 10% SRGF for cell resuspension, 2 ml of growth medium without SRGF were added to reconstitute 5% SRGF concentration in a final volume of approximately 4 ml).

After approximately 12-48 hour from transfection, cell viability, gene expression and transfection efficiency were evaluated.

### EXAMPLE 2 OF THE INVENTION

Identical to the above-mentioned Example 1, with the only difference that cells were expanded in 1.25% vol/vol medium additive rich of human-derived platelet growth factors SRGF (instead of 5%, as in the Example 1).

Moreover, as a consequence, after electroporation step, cells were resuspended in the centrifuge tube using a doubled SRGF concentration (i.e. 2.5% vol/vol, instead of 10%, as in the Example 1).

### EXAMPLE 3 OF THE INVENTION

Identical to the above-mentioned Example 1, with the only difference that cells were expanded in 2.5% vol/vol medium additive rich of human-derived platelet growth factors SRGF (instead of 5%, as in the example 1).

Moreover, as a consequence, after electroporation step, cells were resuspended in the centrifuge tube using a doubled SRGF concentration (i.e. 5% vol/vol, instead of 10%, as in the Example 1).

### EXAMPLE 4 OF THE INVENTION

Identical to the above-mentioned Example 1, with the only difference that cells were expanded in 10% vol/vol medium additive rich of human-derived platelet growth factors SRGF (instead of 5%, as in the example 1).

Moreover, as a consequence, after electroporation step, cells were resuspended in the centrifuge tube using a doubled SRGF concentration (i.e. 20% vol/vol, instead of 10%, as in the Example 1).

### EXAMPLE 5 OF THE INVENTION

Identical to the above-mentioned Example 1, with the only difference that cells were expanded in 20% vol/vol medium additive rich of human-derived platelet growth factors SRGF (instead of 5%, as in the example 1).

Moreover, as a consequence, after electroporation step, cells were resuspended in the centrifuge tube using a doubled SRGF concentration (i.e. 40% vol/vol, instead of 10%, as in the Example 1).

### EXAMPLE 6 (CONTROL)

Identical to the above-mentioned Example 1, with the only difference that cells were expanded without medium additive rich of human-derived platelet growth factors (SRGF); instead 10% vol/vol fetal bovine serum (FBS) was added. Moreover, upon detachment, expanded ASC were placed in the incubator for 4 minutes at 37° C. (instead of 2 minutes, i.e. the incubation time to detach 5% SRGF expanded ASC—see example 1 of the invention). Finally, cells were resuspended in the tube, still using a doubled FBS concentration (i.e. 20% vol/vol, instead of 10% FBS, as during the cell expansion phase) in analogy with the Example 1 of the present invention, where SRGF concentration was doubled when compared to the cell expansion phase.

### Evaluation

Cell samples collected for the present invention, as described in examples 1-5 and in the control example number 6, were analyzed for viability, gene construct expression and transfection efficiency. Viability of transfected cells was comprised between 77±2% (using the C-17 program of the Nucleofector® 2b (Lonza)) and 59±3% (when cells were expanded in presence of 10% FBS). In contrast, the viability of cells expanded in FBS and transfected by both programmes was less than 5%. The following table reports transfection efficiency obtained using the C-17 program on ASC expanded in presence of different concentrations of SRGF (medium additive rich of human-derived platelet growth factors) or FBS (control medium additive).

TABLE 1

| | | Transfection efficiency |
|---|---|---|
| | Additive | (Mean ± S.E.M.) |
| Sample 1 (invention) | 5% SRGF | 50 ± 6% |
| Sample 2 (invention) | 1.25% SRGF | 32 ± 3% |
| Sample 3 (invention) | 2.5% SRGF | 35 ± 7% |
| Sample 4 (invention) | 10% SRGF | 48 ± 4% |
| Sample 5 (invention) | 20% SRGF | 50 ± 3% |
| Sample 6 (control) | 10% FBS | 22 ± 5% |

From the data shown in Table 1, it can be seen that the samples of the invention in which ASC were expanded in presence of SRGF (medium additive rich of intraplatelet growth factors) from 1.25 to 20% vol/vol were characterized by an increased electroporation efficiency when compared to the control samples, where cells were expanded without SRGF but in presence of 10% vol/vol fetal bovine serum (FBS). Cells expanded in presence of 5% SRGF showed the highest electroporation efficiency, equal to the efficiency showed by cells expanded in presence of 10% and 20% of the same additive. Cells expanded with the 2.5% and 1.25% additive showed a reduction in electroporation efficiency, but still remaining superior to the control sample.

Addition of 5% SRGF was shown to be an optimal and safe condition to expand mesenchymal stem/stromal cells derived from adipose tissue (ASC).

Naturally, many modifications and variations of the described preferred embodiments will be evident to those skilled in the art, still remaining within the scope of the invention.

Therefore, the present invention is not limited to the preferred embodiments described, illustrated only by way of non-limiting example, but is defined by the following claims.

1. A method for the modification of mesenchymal stromal stem cells comprising the following phases:
a) seeding said cells on a plastic support in the presence of an appropriate growth-medium promoting their expansion;
b) adding at least one medium additive rich in human-derived platelet growth factors aimed to promote the expansion of said cells;
c) detaching said expanded cells from the plastic surface;
d) adding to said expanded cells a gene construct in order to obtain a mixture; characterized in that said method also comprises the following phases:
e) submitting said mixture to one or more electrical pulses to electroporate said mixture;
f) suspending said electroporated mixture having been submitted to one or more electrical pulses in said growth-medium comprising the additive rich in human-derived platelet growth factors, and transferring the resulting mixture into a suitable container for incubation;
g) resuspending said cell mixture adding an amount of growth-medium containing a percentage of additive rich in human-derived platelet growth factors higher than the same growth-medium used in the previous phase (b);
h) transferring the modified cells to a plastic support for cell culture and incubating;
i) at the end of the incubation, adding to said support a volume of growth-medium devoid of said additive rich in human-derived platelet growth factors to restore the equivalent additive concentration applied in the growth-medium used in said phase (b) for cell expansion.

2. The method according to claim 1 wherein said mesenchymal stromal stem cells are isolated from adipose tissue or other sources such as adult tissues and fetal annexes selected from the group consisting of bone marrow, dental pulp, umbilical cord, Wharton's jelly, and amniotic fluid.

3. The method according to claim 1, wherein said additive rich in human-derived platelet growth factors is obtained from whole blood or platelet-apheresis plasma procedures.

4. The method according to claim 1, wherein said additive rich in human-derived platelet growth factors used in phase b) is added in percentages comprised within a range of 1% to 20% vol/vol, 1.25% to 15% vol/vol, 2.5% to 12% vol/vol, 5% vol/vol.

5. The method according to claim 1, wherein said electrical pulses are of different magnitude and waveform capable of creating a plurality of transient pores in the cell membranes through which said gene construct may permeate the cell.

6. The method according to claim 1, wherein in said phase f) the mixture of cells expanded by means of the growth-medium containing the additive rich in human-derived platelet growth factors is incubated at about 37° C. for a period of less than 60 minutes.

7. The method according to claim 1. wherein said additive rich in human-derived platelet growth factors used in phase g) is added at percentages that are about twice as high as the percentages of the same additive used in phase b) above.

8. Mesenchymal stromal stem cells expanded by a growth-medium containing an additive rich in platelet growth factors, electroporated and genetically modified, and obtained by the method of claim 1.

9. The use of mesenchymal stromal stem cells expanded by a growth-medium containing an additive rich in human-derived platelet growth factors according to claim 8 for cytotoxic treatment against a tumor mass.

10. The use of mesenchymal stromal stem cells expanded by mean of a growth-medium containing an additive rich in human-derived platelet growth factors and processed according to claim 8 to induce expression of trophic, anti-inflammatory, and/or antioxidant factors promoting tissue regeneration/reconstruction.

11. The use of mesenchymal stromal stem cells expanded by a growth-medium containing an additive rich in human-derived platelet growth factors and processed according to claim 8 for the treatment of pulmonary fibrosarcoma and/or glioblastoma.

12. A composition comprising mesenchymal stromal stem cells expanded by a growth-medium containing an additive rich in human-derived-platelet growth factors and processed according to claim 8.

13. The method according to claim 1, wherein in said phase f) the mixture of cells expanded by means of the growth-medium containing the additive rich in human-derived platelet growth factors is incubated at about 37° C. for about 20 minutes.

14. The method according to claim 1, wherein said additive rich in human-derived platelet growth factors used in phase g) is added at percentages that are around 10% vol/vol.
